(19) United States
(12) Patent Application Publication
(10) Pub. No.: US 2010/0307513 A1
(43) Pub. Date: Dec. 9, 2010

(54) COMPONENT FOR AFFIXING AN ARTICLE OF MEDICAL-TECHNICAL NATURE TO SKIN

(75) Inventors: Anna Svensby, Vastra Frolunda (SE); Tomas Fabo, Molnlycke (SE)

Correspondence Address: YOUNG & THOMPSON, 209 Madison Street, Suite 500, Alexandria, VA, 22314, US

(73) Assignee: MOLNLYCKE HEALTH CARE AB, Goteborg (SE)

(21) Appl. No.: 12/671,781

(22) PCT Filed: Sep. 6, 2007
(86) PCT No.: PCT/SE07/50514

§ 371 Date: Aug. 31, 2010

(51) Int. Cl. A61B 19/08; B32B 7/12; A61F 5/445; A61M 25/02

(52) U.S. Cl. 128/849; 428/354; 604/332; 604/180

(57) ABSTRACT

A component (2) for affixing an article of a medical-technical nature to skin or included in a medical article (1) designed to be secured to skin, includes a layer of support material (4) which on its underside, i.e. the side directed towards the skin during use of the medical-technical article, is coated with a layer of adhesive (3). The component (2) has, on its top face, a fastening area (5) for the article or element of the article, which fastening area is located at a distance from at least two opposite edges of the component. Moreover, the adhesive (3) is a skin-friendly, soft, pressure-sensitive adhesive which, in tests of creeping removal, has a removal speed of greater than 0.05 mm/min at a load of 0.2 N/25 mm.

### TECHNICAL FIELD

The present invention relates to a component for affixing an article of a medical-technical nature to skin or included in a medical article designed to be secured to skin, said component comprising a layer of support material which on its underside, i.e. the side directed towards the skin during use of the medical-technical article, is coated with a layer of adhesive.

### BACKGROUND TO THE INVENTION

Self-adhesive plasters are often used to secure articles of a medical-technical nature to skin or to keep the articles bearing against the skin. Examples of such articles are surgical drapes or surgical covers, infusion hoses, etc. The self-adhesive plaster is often a component included in the medical-technical article, e.g. a component in a stoma dressing.

It is of very great importance that such articles of a medical-technical nature do not come loose during use, and that they instead remain securely in place after application. To ensure this is the case, it has been customary to choose plasters with hard, strongly adhering adhesives, for example acrylate adhesive. However, hard adhesives have the disadvantage of affixing so hard to the skin that, when they are removed, they take off skin cells with them. In addition to the pain that arises upon removal of such plasters, the skin's barrier function is also temporarily impaired by removal of skin cells, and there is also a risk that sensitive skin, e.g. newly formed skin, will be seriously damaged during said removal. The fact that the surface of the adhesive is coated with a large number of skin cells after it has been removed means that the adherence of the adhesive layer is considerably impaired, and plasters with hard adhesives can seldom be secured more than once.

From SE-C2-510 907, WO 03/079919 A1 and PCT/SE2006/000025, it is known to instead use pressure-sensitive, soft adhesives which are very gentle on the skin and which, when removed, basically only take dead skin cells with them. The reason for the skin-friendly properties of these soft adhesives is of course that the adhesive does not adhere so hard to the skin, while the softness of the adhesive layer means that the adhesive, in contrast to hard adhesives, has a very good ability to follow the irregularities of the skin. This means that the contact surface area for the adhesive layer is much greater than for hard adhesives, which in turn means that it is still possible to achieve a sufficiently high overall adherence of a medical-technical article to the skin. The low adherence of soft adhesives to skin cells means that they can be removed from the skin without to any appreciable extent removing healthy skin cells with them. Besides the fact that this means they can be removed without causing the user pain, the absence of skin cells on a removed dressing provided with a layer of soft adhesive means that such a dressing can be reapplied with essentially the same adherence capacity.

A weakness of self-adhesive articles secured to the skin by means of skin-friendly soft adhesive is that, despite having good adherence under short-term loads, they often have inadequate adherence under long-term loads, even low loads. For example, in articles where the fastening is loaded by the force of gravity of the secured article, for example stoma bags or surgical drapes, it has been found that after a certain time, which can be less than one hour, the fastening comes loose on account of a slow creeping removal.

The object of the present invention is to eliminate this weakness of articles secured to skin by means of pressure-sensitive, soft adhesives.

### DISCLOSURE OF THE INVENTION

This object is achieved by means of a component for affixing an article of a medical-technical nature to skin or included in a medical article designed to be secured to skin, said component comprising a layer of support material which on its underside, i.e. the side directed towards the skin during use of the medical-technical article, is coated with a layer of adhesive, characterized in that the component has, on its top face, a fastening area for the article or element of the article, which fastening area is located at a distance from at least two opposite edges of the component, the adhesive being a skin-friendly, soft, pressure-sensitive adhesive which, in tests of creeping removal, has a removal speed of greater than 0.05 mm/min at a load of 0.2 N/25 mm.

The fastening area is preferably located at a distance from all the edges of the component.

According to a preferred embodiment, the adhesive, in tests of creeping removal, has a removal speed of greater than 0.5 mm/min at a load of 0.4 N/25 mm.

Moreover, the adhesive is advantageously a skin-friendly, soft, pressure-sensitive adhesive that has an adherence to skin of 0.2-4 N/25 mm, and a softness of 10-22 mm. The support layer is preferably a plastic film, advantageously a polyurethane film with a thickness of 10-50 micrometres.

The fastening area is preferably located at a distance of at least 5 mm from at least those opposite edges of the component that are loaded by tensile and/or shearing forces during use of the article.

In one variant of the invention, a surgical drape or a surgical cover is secured to the component.

In another variant, the component is a component of a stoma dressing.

In a third variant, the component comprises a member for securing a hose or other articles of a medical-technical nature to its top face. The securing member can be formed by male or female parts of a mechanical securing element.

### BRIEF DESCRIPTION OF THE DRAWINGS

The invention will now be described with reference to the attached figures, of which:

FIG. 1 shows a schematic sectional view of a surgical drape fastened to a patient in accordance with the prior art,

FIG. 2 shows a schematic sectional view of a surgical drape fastened to a patient in accordance with an embodiment of the invention,

FIG. 3 is a schematic representation of part of the view from FIG. 2, on an enlarged scale,

FIG. 4 shows a schematic plan view of an annular component for fastening an article of a medical-technical nature to the skin of a user,

FIG. 5 shows a schematic plan view of a component for securing a hose or a surgical element to the skin of a patient,

FIG. 6 shows a schematic cross section along the line VI-VI in FIG. 5,

FIG. 7 is a schematic illustration of a method for measuring adherence to skin,

FIGS. 8 and 9 are schematic illustrations of a method for measuring softness,

FIGS. 10 and 11 are schematic illustrations of a method for measuring creeping removal of adhesive,

FIG. 12 shows a diagram of the creeping removal for different adhesives, and

FIGS. 13 and 14 illustrate a measurement of the creeping removal of adhesive, with a distribution of the load in accordance with the present invention.

### DESCRIPTION OF EMBODIMENTS

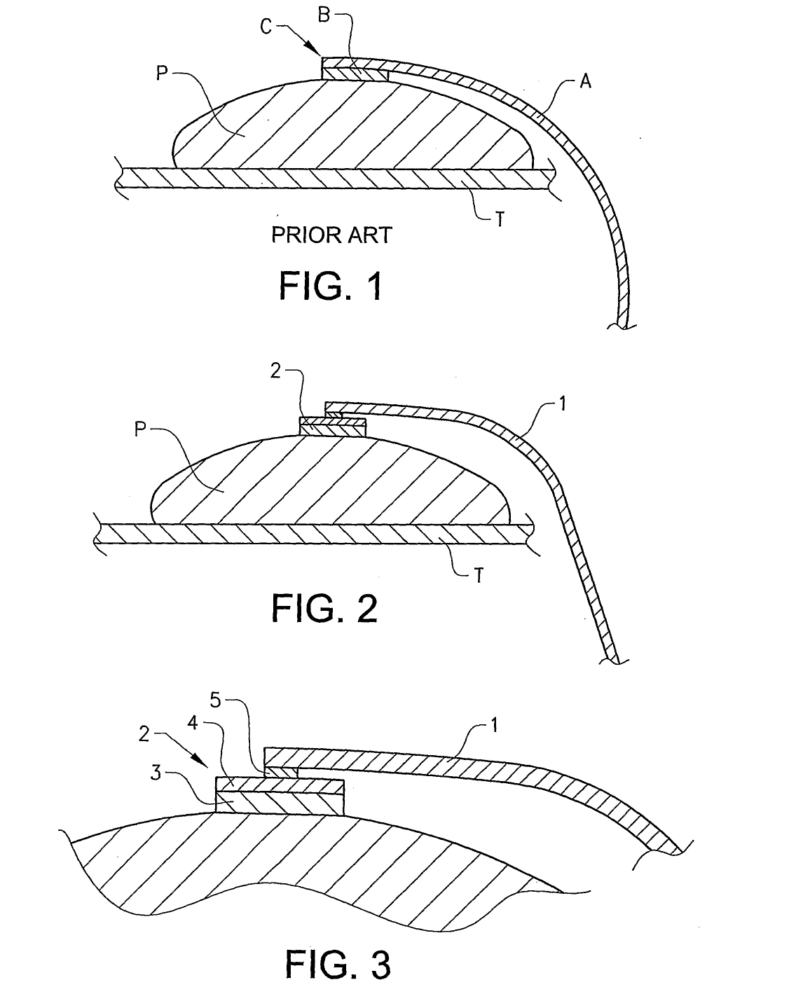

FIG. 1 shows a surgical drape A according to the prior art arranged on a patient P, who is lying on an operating table T. Along its upper edge, this drape has an adhesive coating B, which secures the drape to the patient. This fastening is continuously loaded by the weight of the drape and is dimensioned to take up this load, which normally increases with time, on account of the fact that the drape takes up liquid either by absorption or by way of liquid-collecting pouches or the like arranged at the lower edge of the drape. In recent times, the use of soft, skin-friendly adhesives has been proposed for the coating B, on the one hand because these do not take skin cells with them when being removed, and on the other hand because they offer the possibility of extremely good sealing against the skin. However, it has been found that, with soft skin-friendly adhesives, there is a risk of the drape having a tendency to come loose under a long-term load, on account of slow creeping removal.

When the adhesive coating B of the drape A in FIG. 1 is subjected to the weight of the drape, the forces between skin and adhesive are concentrated at the edge C of the contact face of the adhesive coating B. Although the tensile force from the drape A is for the most part distributed as shearing force in the adhesive coating B, relatively small tearing forces do occur. There is then a risk of the load becoming locally higher than the adherence of the adhesive, and of the connection between skin and adhesive coming loose at the edge C, which means that the loading forces are shifted slightly in from the edge. This process then continues such that the drape comes loose as a result of slow and gradual removal.

It will be appreciated that the abovementioned sensitivity of soft, skin-friendly adhesives to loads concentrated on one edge of the contact face between skin and adhesive also applies to articles other than surgical drapes and of course also applies to short-term loads concentrated on one edge of the contact face.

Corresponding processes of removal may take place at other edges, depending on other loads that arise during use of the drape, including lifting forces.

The present invention aims primarily to solve the problem of the abovementioned creeping removal.

Since soft, skin-friendly adhesives can, as has been mentioned above, follow the contours of the skin extremely well, not only is a large surface area of contact obtained by comparison with hard adhesives, but also an underpressure effect that increases the adherence of the dressing to the skin. This effect is achieved upon application by virtue of the fact that all the air under the contact surface can be pressed out when an article provided with such an adhesive coating is applied to the skin. The contact surface can be likened to mechanical suction plugs that are based entirely on the principle of creating an underpressure during loading. This effect is best ensured if the adhesive coating is of such a type that it also provides a seal against so-called microleakage, i.e. prevents air from penetrating under the contact surface between adhesive and skin, through folds or cracks in the skin.

FIGS. 2 and 3 (larger scale) show schematic illustrations of an embodiment of a surgical drape 1 provided with a component 2 according to the invention. The component 2 is composed of a support 4, coated with a layer of soft, skin-friendly adhesive 3. The edge of the drape 1 is secured to the support 4 by a narrow adhesive seam 5, which extends centrally on the top face of the component 2 at a distance from what are, in FIGS. 2 and 3, the left-hand and right-hand edges of the component 2. The seam 5 can comprise any suitable adhesive, for example an acrylate adhesive. It is of course also possible to secure the edge of the drape 1 to the support 4 by means of other types of seams, for example a weld seam.

The load of the drape 1 is concentrated at the centre of the component 2, which extends along the edge of the drape. The load will then be distributed across an area under the seam 5 and will decrease in the direction of the left-hand and right-hand edges in FIGS. 2 and 3. This has the result that tearing forces and shearing forces at the edges are less than if the drape were to be secured to the entire top face of the component 2, as in the prior art shown in FIG. 1. This reduces the risk of the drape coming loose as a result of the abovementioned creeping removal, which starts from one of the edges of the fastening. This placement of the fastening of the drape against the component 2 also increases this component's resistance to high short-term loads, for example in the normal direction of the skin. Such loads may lift central parts of the component 2 from the skin, but, since no air penetrates via the edges, the underpressure created will again suck these central parts against the skin as soon as the load ceases. Since the soft, skin-friendly adhesive does not pull off skin cells with it when the central parts lift from the skin, the component will have to a large extent the same high degree of adherence to the skin, after having been sucked firmly by the underpressure, as it does after a first application.

Although in many cases it may suffice for the component 2 to end at the side edges of the drape 1, i.e. the edges that lie in planes parallel to the plane of the paper in FIGS. 2 and 3, it is expedient to allow the component 2 to extend past these edges. This reduces the risk of the side edges of the component coming loose after short-term or long-term loading of the side edges of the drape. In other types of articles of a medical-technical nature, where long-term loads may load several edges, it is necessary that the fastening of the medical-technical article to the top face of the component is located at a distance from all its edges. The distance from the edges subjected to loading must be 5 mm, preferably 10 mm, most preferably 15 mm or more.

For the component 2 to be able to function in the intended manner, it is important that the support 4 is not so stiff that it transmits the central load directly to the edges of the component, and instead the forces are to be introduced into and distributed within the underlying adhesive layer. In this way, the forces are concentrated on the central part of the adhesive coating and decrease in the direction towards the edges, which considerably reduces the risk of the component 2 coming loose. To ensure that the force is taken up in this desired way in the adhesive coating, the support 4 must be flexible, stretchable and also advantageously elastic, such that it recovers its original configuration after stretching. These properties of the support also mean that the entire component 2 can follow the irregularities of the skin and thus prevent air pockets from forming between adhesive and skin after application of the component. The support can be a plastic film, e.g. a polyurethane film with a thickness of 10-50 micrometres. Other plastic materials that can be used are polyester and polyethylene. The thickness of the plastic film is preferably less than 50 micrometres, more preferably 10-30 micrometres.

Suitable soft, skin-friendly adhesives suitable for use according to the invention can be composed, for example, of an addition-cured RTV (Room Temperature Vulcanizing) silicone system which, after admixture, crosslinks and forms a self-adhesive elastomer. Examples of RTV addition-cured silicone systems are given in EP 0 300 620 A1 which describes gel-forming compositions composed of an alkenyl-substituted polydiorganosiloxane, an organosiloxane containing hydrogen atoms linked to some of the silicone atoms, and also a platinum catalyst.

Wacker SilGel 612 is a commercially available RTV silicone system. This is a two-component system. The softness and degree of adherence of the elastomer which is formed can be varied by varying the proportions of the two components A:B from 1.0:0.7 to 1.0:1.3.

Examples of other soft silicone elastomers that adhere to dry skin are NuSil MED-6340, NuSil MED3-6300 and NuSil MED 12-6300 from NuSil Technology, Carpintieria, Ga., USA, and Dow Corning 7-9800 from Dow Corning Corporation, Midland, USA.

Other soft skin-friendly adhesives can also be used with the present invention, for example thermal adhesives such as Dispomelt® 70-4647 from National Starch and Chemical Company, Bridgewater, N.J., USA.

The component 2 functions best if the adhesive coating also provides a seal against microleakage, i.e. prevents air from penetrating under the adhesive coating via cracks in the skin, skin folds, or other irregularities in the skin. Such ingress of air can reduce or even eliminate the above-mentioned underpressure effect of an applied document. For a skin-friendly adhesive, it has surprisingly been found that the abovementioned risk of leakage can be eliminated or at least significantly reduced if the adhesive is sufficiently soft and has a sufficiently high weight per unit of area. The adhesive coating 3 should therefore have a softness of 10-22 mm and a weight per unit of area of 50 g/m² or more. The adhesive coating will preferably be leaktight in accordance with the MHC Leakage Test with a groove depth of 75 micrometres. The MHC Leakage Test is described more closely in patent application SE 0500061-7, to which reference may be made for more details.

Since the properties of the skin vary from person to person, the ability of the adhesive coating to adhere to the skin of different patients will of course also vary. The adherence also depends on the thickness of the soft adhesive and on the mechanical properties of the support layer. The standard methods available today for measuring adherence use plates of various types, for example of steel or glass, and do not provide values that are relevant to measuring adherence to skin. The values for the adherence of an adhesive to skin will, as has been stated, be measured by means of a method that is illustrated schematically in FIG. 7 and that has been developed by the applicant.

Strips of a self-adhesive component, whose adherence to skin is to be measured, are cut to a size of 25×125 mm. It should be noted that all the strips are also provided with a support layer on the back of the film dressing. (The function of this support layer is to stiffen the strips during application to the skin.) Thereafter, the strips are placed on the skin on the back of healthy volunteers. The strips are carefully pressed with a finger, and then the support layer on the back of the strips is removed. Finally, the strips are pressed firmly against the skin for 3 seconds with the aid of a sponge made of foamed plastic (42×182 mm, thickness=48 mm) glued firmly onto a steel plate (50×200 mm, thickness=1 mm). The pressing force is estimated at 6 kN/m². The strips are left on the skin for 2 minutes. Thereafter, the strips are removed at a speed of 25 mm/second, and the removal force F1 is measured. The removal angle, i.e. the obtuse angle formed between the surface of the skin and the removed part of the strip, is to be 135°. The adherence of the strip to skin is the average of the force F1.

Adhesives that can be used in components according to the invention must have an adherence, according to this method, of at least 0.2-4 N/25 mm. The adherence is preferably 1-2.5 N/25 mm.

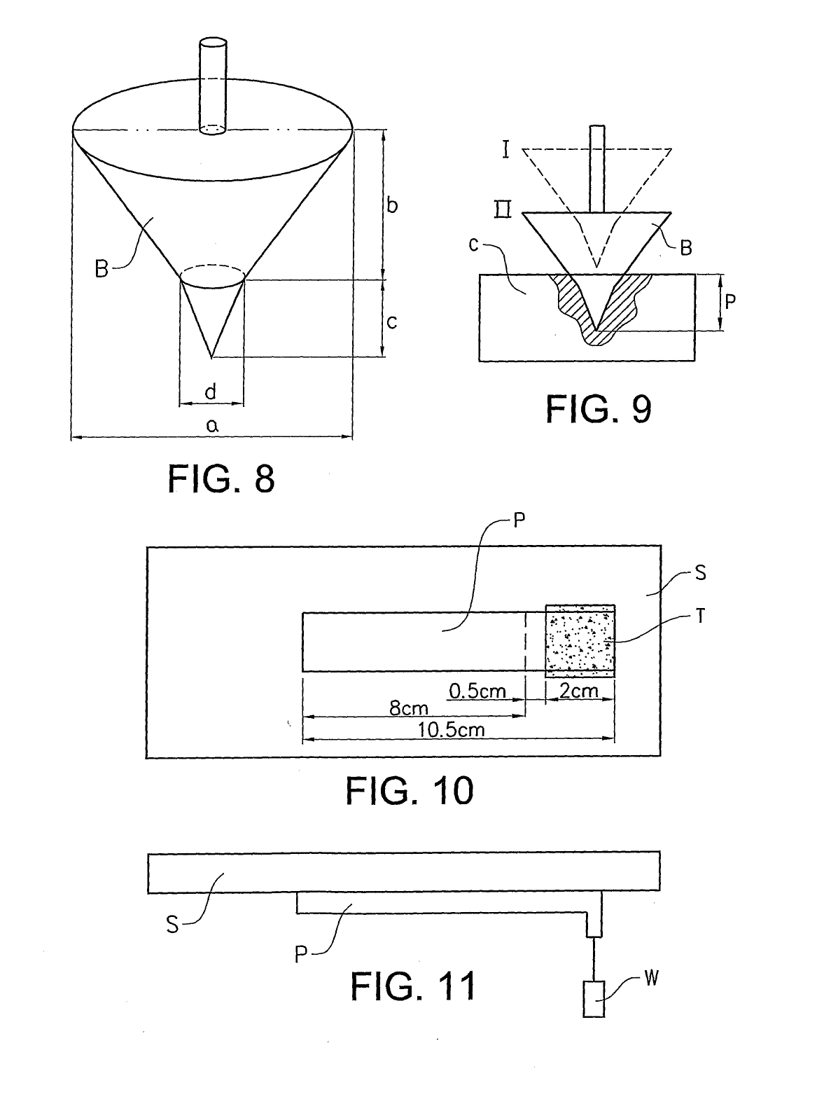

Adhesives according to the present invention must have a softness that exceeds 10 mm measured by a method based on ASTM D 937 and ASTM D 51580. Certain modifications have been made and are described below. FIGS. 8 and 9 illustrate this modified method for measuring softness of an adhesive by letting a cone B with a weight of 62.5 g penetrate by gravity into a 30 mm thick sample C of the adhesive whose softness is to be determined. The sample is produced by filling a cylindrical glass container, which has an internal diameter of 60 mm and an internal height of 35-40 mm, with adhesive up to a height of 30 mm. For a silicone elastomer, non-cured silicone prepolymer is poured into the container and is then crosslinked to an elastomer in the glass cylinder. The cone that is used is shown in FIG. 8 and has the following dimensions: a=65 mm, b=30 mm, c=15 mm and d=8.5 mm. When carrying out the method for measuring softness, the cone B is first lowered to a position I, which is shown by broken lines in FIG. 9 and in which the tip of the cone just brushes the surface of the sample C. The cone B is then released, such that it can penetrate into the sample C by force of gravity. The number of mm by which the tip of the cone B has penetrated into the sample C after 5 seconds is measured and constitutes the penetration value P, which is greater, the softer the sample. The penetration value P constitutes the measurement of softness used in the present invention. The method is carried out using a Penetrometer PNR 10 from Sommer & Runge KG, Germany.

The applicant has developed a test method for measuring the creeping removal of an adhesive. Note that this method of determining the creep properties of an adhesive applies only to samples whose surface is coated completely with adhesive.

Samples P measuring 25×105 mm are cut out from material coated with the adhesive that is to be tested.

A scratch-free steel plate S (according to ASTM A 666-94 A, 50×200 mm) is washed three times with a lint-free absorbent material soaked with n-heptane. Finally, a last wash is performed with acetone instead of n-heptane. The steel plate is then left to dry for at least 10 minutes, but for not longer than 10 hours.

The sample P is strengthened at one end with the aid of a strong tape T. A piece of this tape, said piece measuring 4 cm in length, is weighed and then folded and secured around the end of the sample, as is shown schematically in FIG. 10. For a sample P of adhesive-coated film, the latter is placed on the plate with the adhesive directed towards the steel plate, after which the tape is folded over one edge of the sample, and the stiffening layer that facilitates application of the film is carefully removed. A hole is made in the tape and through the end of the sample to allow a weight W to hang free. It is important that the sample is placed on the steel plate carefully so as to ensure that no pressure is exerted on the sample. For samples without a stiffening layer that is to be taken off, the tape can be secured to the end of the sample before the sample is secured to the steel plate.

Thereafter, a piece of polyurethane foam (L00562-6, 1.6 mm from Rynel Inc. Boothbay, Me., USA) is placed over the sample on the steel plate, and the sample is secured firmly to the plate by means of a roller (width=45 mm, weight=2040 g, r=47 mm) being rolled to and fro once on the piece of foam, at a speed of 5 mm/second. Thereafter, the sample is left to rest for 1 hour.

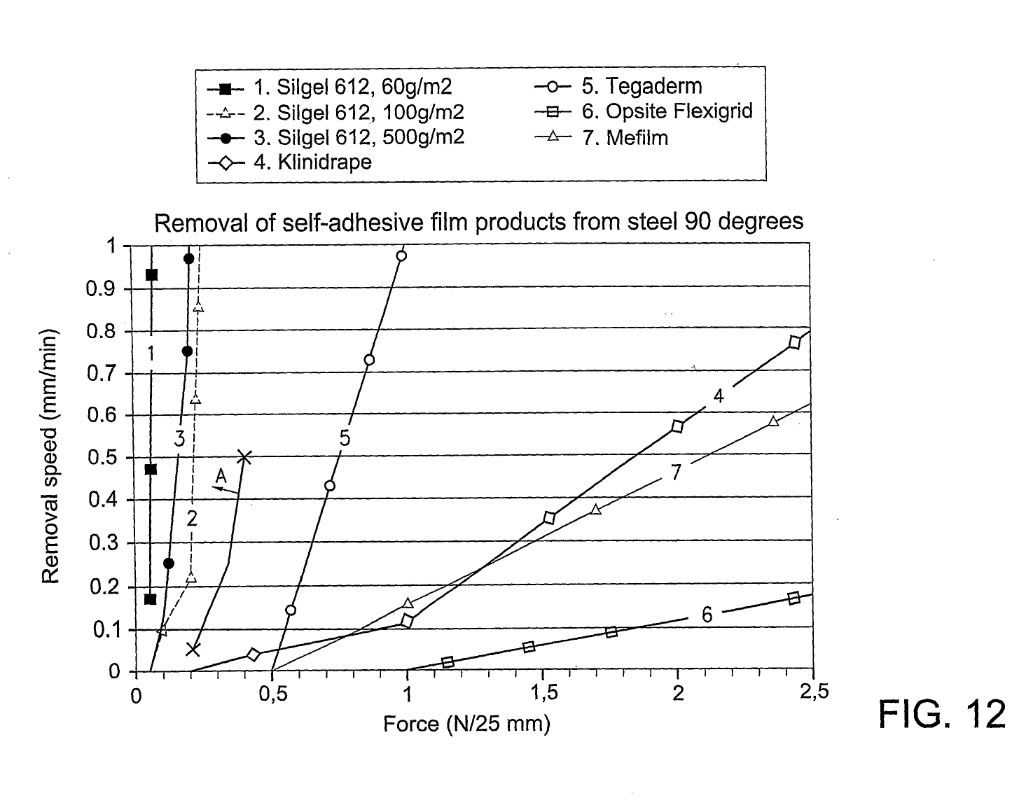

After 1 hour has passed, a weight W is secured in the tape-containing end of the sample P, and the end is folded out such that the weight hangs down straight as shown in FIG. 11, and the zero point is marked on the steel plate. The zero point is placed 2.5 cm from the end of the piece of tape, such that 8 cm of the sample is secured to the steel plate and 2.5 cm hangs down with weight, as shown in FIG. 11. The time starts to be taken and, depending on the weight and type of adhesive, the time interval is varied and marked on the steel plate. The markings are then calculated to give the removal speed of the sample in mm/min.

Products and materials tested by the method above

Polyurethane film 20 μm coated with silicone gel (Silgel 612 from Wacker
Chemie GmbH Germany) with a softness of 15 mm and a weight per
unit of area of 60 g/m²
Polyurethane film 20 μm coated with silicone gel (Silgel 612 from Wacker
Chemie GmbH Germany) with a softness of 15 mm and a weight per
unit of area of 100 g/m²
Polyurethane film 20 μm coated with silicone gel (Silgel 612 from Wacker
Chemie GmbH Germany) with a softness of 15 mm and a weight per
unit of area of 500 g/m²
Klinidrape ® from Mölnlycke Health Care AB, Sweden (samples taken
from the self-adhesive edges of the product)
Tegaderm ™ from 3M Health Care, USA
Opsite Flexigrid ™ from Smith & Nephew Medical Limited, England
Mefilm ™ from Mölnlycke Health Care
AB, Sweden

The abovementioned products were tested with different weights corresponding to forces of 0.05, 0.1, 0.2, 0.5, 1 and 2.5 N/25 mm.

The results of these tests are shown in FIG. 12 and in Table 1 below.

TABLE 1
Removal speed 90 degrees from steel

| | Force | Force | Force | Force | Force | Force |
|---|---|---|---|---|---|---|
| | N/25 mm | N/25 mm | N/25 mm | N/25 mm | N/25 mm | N/25 mm |
| | 0.05 | 0.1 | 0.2 | 0.5 | 1 | 2.5 |
| | Speed | Speed | Speed | Speed | Speed | Speed |
| Material | mm/min | mm/min | mm/min | mm/min | mm/min | mm/min |
| SiliGel | 0.17 | 1.87 | 28 | 1000 | 1000 | 1000 |
| 60 g/m2 | | | | | | |
| SiliGel | 0 | 0.1 | 0.22 | 4.8 | 30.2 | 1000 |
| 100 g/m2 | | | | | | |
| SiliGel | 0 | 0.13 | 0.75 | 8.9 | 87 | 1000 |
| 500 g/m2 | | | | | | |
| Klinidrape | 0 | 0 | 0 | 0.05 | 0.11 | 0.79 |
| Tegaderm | 0 | 0 | 0 | 0 | 0.98 | 1000 |
| Opsite | 0 | 0 | 0 | 0 | 0 | 0.17 |
| Flexigrid | | | | | | |
| Mefilm | 0 | 0 | 0 | 0 | 0.15 | 0.62 |

In the test performed, the adhesive is thus subjected to a local linear load. The result of the tests can thus be said to constitute a measurement indicating the local adherence of the tested adhesive. As is evident from the test, the soft and skin-friendly adhesives, which according to the invention are suitable for securing to skin, come loose from the substrate at relatively low, long-term loads of 0.05-0.2 N/25 mm, while the adhesive of the other tested products remained secured to the substrate. This confirms the theory that it is the low local adherence to skin of the soft, skin-sensitive adhesives that to a large extent contributes to the skin friendliness and other good properties of such adhesives. The test carried out can be said to constitute a kind of measure of the local adherence of an adhesive.

For an adhesive to be considered soft and skin-friendly according to the invention, it must lie to the left (see arrow A) of the line drawn between two crosses in FIG. 12. Thus, the removal speed must be greater than 0.05 mm/min at a load of 0.2 N/25 mm and greater than 0.5 mm/min at a load of 0.4 N/25 mm.

To ascertain the effects of the present invention, the following test was carried out.

A scratch-free steel plate S (according to ASTM A 666-94 A, 50×200 mm) is washed three times with a lint-free absorbent material soaked with n-heptane. Finally, a last wash is performed with acetone instead of n-heptane. The steel plate is then left to dry for at least 10 minutes, but for not longer than 10 hours. Samples P of the same material as above, of size 25×105 mm, are secured with the pressure-sensitive adhesive to the steel plate in the same way as has been described above.

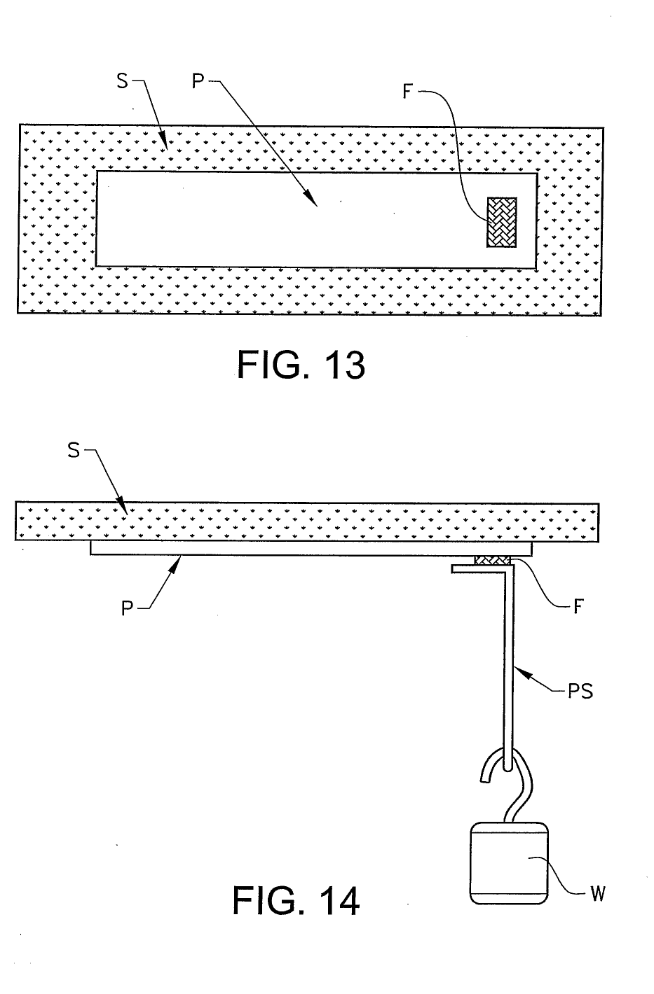

A pad F that is adhesive on two sides and consists of ca. 1 mm thick foamed plastic measuring 10×15 mm is secured to one end of the sample P, such that three of the four edges of the pad F lie 5 mm inside the edges of the sample. The position of the corners of the sample is marked on the steel plate S with a felt-tip pen, such that any removal of the sample from the steel plate and/or any lateral shifting of the sample can be read off with an accuracy of 0.5 mm. The test is illustrated schematically in FIGS. 13 and 14.

A strip of paper PS loaded with the same weights as in the test according to Table 1 is secured to the pad F. The distance that the corners of the sample have shifted under loading for a period of 30 min is read off. The result was that no removal or shifting took place, either for the samples coated with SilGel 612 or for the other samples. A marked improvement in the resistance to long-term creeping load of the samples coated with SilGel 612 was therefore able to be confirmed.

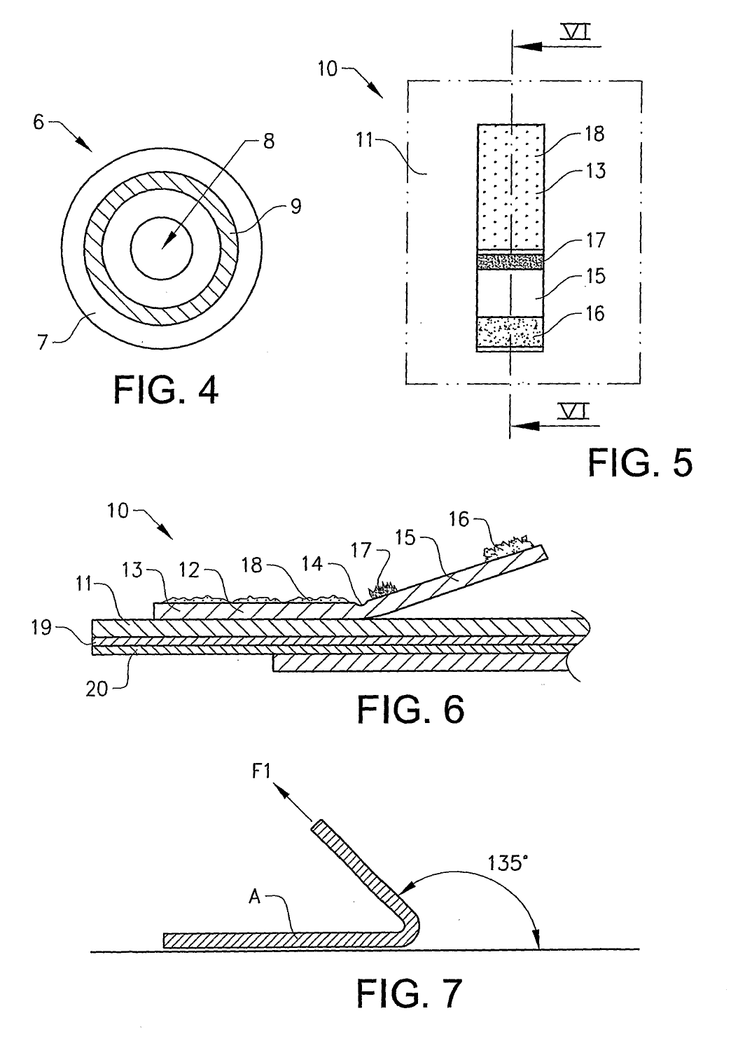

FIG. 4 shows a plan view of a component 6 included in a stoma dressing.

The component 6 comprises a circular support 7 with a hole 8 at the centre for passage of a stoma. On its underside, i.e. the side directed towards the user's skin during use of the component, the support 7 is provided with a coating of a soft and skin-friendly adhesive. On the top face of the support 7, the component 6 has an annular fastening area 9 (indicated by broken lines in the figure) for the actual stoma bag (not shown in the figure). The fastening of the bag can involve an adhesive connection, which makes it easier to change the bag, but can also involve a fixed connection in the form of a thermal weld seam. In order to avoid a concentration of forces at the component's outer or inner edge in this case too, the fastening area must be situated at a distance from both edges of the component, by a distance of at least 5 mm. In such a component, the adhesive coating not only has to prevent entry of air but also has to ensure that liquid from the stoma does not pass out onto the skin surrounding the component 6. It is therefore of great advantage if the adhesive coating has such a combination of softness and weight per unit area that it also provides a seal against microleakage in order to ensure this function. The support materials and adhesives that are suitable for the component 2 shown in FIGS. 2 and 3 are also suitable in this application of the invention.

It will be noted that when the fastening area is described as being located “at a distance from at least two opposite edges of the component”, this applies also to components with a round or oval shape, in which one half of the contour represents an edge and the other half an opposite edge.

FIGS. 5 and 6 show a third embodiment of the invention comprising a component 10. This component has a support 11, on whose top face a securing element 12 for a hose or a surgical instrument is secured, preferably by means of an adhesive dressing. The securing element 12 comprises a lower part 13 which on its underside is secured to the top face of the support 11, and an upper part 15 which can be folded in across the lower part 13, e.g. by means of a hinge 14. The upper part 15 has hook members 16, 17 on the side facing the lower part when the upper part is in the folded-in state, and the lower part has on its top face loop members 18 which can cooperate with the hook members 16, 17 in order to releasably lock the lower and upper parts to each other. The loop members could of course be arranged instead on the upper part and the hook members on the lower part.

The upper and lower parts 13, 15 can be made of a plastic material, a nonwoven (NW) or a textile material, or a laminate of plastic and NW or plastic and textile material. In cases where NW or textile material is included in or constitutes the upper and lower parts, no separate loop members are needed, and instead it is possible to use hook members that are able to cooperate with this material.

On its underside, the support 11 has an adhesive coating 19 which is covered by release paper 20, which provides protection for the adhesive coating before use and which is removed prior to application of the component. The support materials and adhesives that are suitable for the component 2 shown in FIGS. 2 and 3 are also suitable in this application of the invention. In this embodiment too, the fastened lower part 13 of the securing element 12 must be located at a distance of at least 5 mm from the edges of the component 10.

The components 2 and 6 according to the embodiments described with reference to FIGS. 2-4 are also preferably provided with release paper or similar protective layers.

The invention is intended to be applicable to all components for fixing an article of a medical-technical nature to skin or included in a medical article designed to be secured to skin, which has or comprises, on its top face, elements for supporting a load, and it is not limited to the embodiments cited above.

The materials included in the components according to the invention are sterilizable.

The described embodiments can of course be modified within the scope of the invention. For example, the component shown in FIG. 4 does not have to be circular, but instead can be rectangular, quadrilateral, octagonal, etc., and the component shown in FIGS. 5 and 6 does not have to be rectangular and instead can be circular. Moreover, the securing element on the top face of the embodiment shown in FIGS. 5 and 6 can be designed in another way, e.g. the hook and loop members could be allowed to extend across the whole surface of the upper and lower part, respectively. Securing elements other than hook and loop members could also be used, e.g. glue or press-studs. In addition, it may be advantageous for at least the upper part to be made of an elastic material. Therefore, the invention is limited only by the content of the attached patent claims.

1. Component (2) for affixing an article of a medical-technical nature to skin or included in a medical article (1) designed to be secured to skin, said component comprising a layer of support material (4) which on its underside, i.e. the side directed towards the skin during use of the medical-technical article, is coated with a layer of adhesive (3), characterized in that the component (2) has, on its top face, a fastening area (5) for the article or element of the article, which fastening area is located at a distance from at least two opposite edges of the component, the adhesive (3) being a skin-friendly, soft, pressure-sensitive adhesive which, in tests of creeping removal, has a removal speed of greater than 0.05 mm/min at a load of 0.2 N/25 mm.

2. Component according to claim 1, in which the fastening area is located at a distance from all the edges of the component.

3. Component according to claim 1, in which the adhesive (3), in tests of creeping removal, has a removal speed of greater than 0.5 mm/min at a load of 0.4 N/25 mm.

4. Component according to claim 1, in which the adhesive (3) has an adherence to skin of 0.2-4 N/25 mm, a softness of 10-22 mm, and a weight per unit of area of at least 50 g/m².

5. Component according to claim 1, in which the support layer (4) is a plastic film.

6. Component according to claim 5, in which the plastic film (4) is a polyurethane film with a thickness of 10-50 micrometres.

7. Component (2) according to claim 1, in which the fastening area (5) is located at a distance of at least 5 mm from at least those opposite edges of the component (2) that are loaded by tensile and/or shearing forces during use of the article.

8. Component (2) according to claim 1, in which a surgical drape (1) or a surgical cover is secured to the component.

9. Component (6) according to claim 1, which is included as a component (6) in a stoma dressing.

10. Component (10) according to claim 1, which comprises a member (12) for securing a hose or other articles of a medical-technical nature to the top face of the component (10).

11. Component according to claim 10, in which the securing member is formed by male or female parts of a mechanical securing element.

12. Component according to claim 2, in which the adhesive (3), in tests of creeping removal, has a removal speed of greater than 0.5 mm/min at a load of 0.4 N/25 mm.

13. Component according to claim 2, in which the adhesive (3) has an adherence to skin of 0.2-4 N/25 mm, a softness of 10-22 mm, and a weight per unit of area of at least 50 g/m².

14. Component according to claim 3, in which the adhesive (3) has an adherence to skin of 0.2-4 N/25 mm, a softness of 10-22 mm, and a weight per unit of area of at least 50 g/m².
